(19) United States
(12) Patent Application Publication
(10) Pub. No.: US 2023/0248137 A1
(43) Pub. Date: Aug. 10, 2023

(54) STETHOSCOPE HOLDER

(71) Applicant: Motivationmed Inc, New Hyde Park, NY (US)

(72) Inventor: Jonathan Jagroop, New Hyde Park, NY (US)

(21) Appl. No.: 18/133,788

(22) Filed: Apr. 12, 2023

Related U.S. Application Data
(63) Continuation-in-part of application No. 29/824,139, filed on Jan. 21, 2022.
(60) Provisional application No. 63/329,980, filed on Apr. 12, 2022.

(51) Int. Cl. A45F 5/02

(52) U.S. Cl. CPC A45F 5/021; A45F 2005/025; A45F 2200/05; A61B 50/20

(57) ABSTRACT

A stethoscope holder includes a tubing holder and a chestpiece holder. The chestpiece holder is located in a first direction side of the tubing holder. The chestpiece holder includes first and second tubing catchers. The chestpiece holder is provided with a gap between the first and second tubing catchers in a second direction perpendicular to the first direction.

### CROSS-REFERENCE TO RELATED APPLICATIONS

This application claims the benefit of U.S. Provisional Application No. 63/329,980, filed on Apr. 12, 2022, and is a continuation-in-part of U.S. Design Application No. 29/824,139, filed on Jan. 21, 2022, the contents of each of which are incorporated herein by reference.

### FIELD

The present disclosure generally relates to a stethoscope holder.

### BACKGROUND

Stethoscopes are tools for evaluating patients' health. Stethoscopes have been used for decades to examine and evaluate patients' health. Medical professionals such as doctors, nurses, nurse practitioners and other medical personnel often carry a stethoscope as part of checking on patients' health. Typically, stethoscopes have multiple parts, such as a flexible tubing, a chestpiece, and a pair of ear temples. The chestpiece may have a bell, which can be placed around the patient's body. The tubing may be designed to extend a substantial distance between the bell and the pair of ear temples.

Medical practitioners frequently suffer from neck pain from wearing a stethoscope around their neck. Thus, the inventor has recognized that there is a need for reducing or avoiding neck pain due to the wearing of a stethoscope around their neck.

The description provided in the background section should not be assumed to be prior art merely because it is mentioned in or associated with the background section. The background section may include information that describes one or more aspects of the subject technology.

### SUMMARY

One embodiment of the present disclosure may provide a stethoscope holder. The stethoscope holder may include a tubing holder and a chestpiece holder. The chestpiece holder may be located in a first direction side of the tubing holder. The chestpiece holder may include first and second tubing catchers. The chestpiece holder may be provided with a gap between the first and second tubing catchers in a second direction perpendicular to the first direction.

Another embodiment of the present disclosure may provide a method of using a stethoscope holder. The method may include placing, in a gap of the stethoscope holder, a tubing of the stethoscope holder. The stethoscope holder may include: a tubing holder; and a chestpiece holder located in a first direction side of the tubing holder. The chestpiece holder may include first and second tubing catchers. The gap may be located between the first and second tubing catchers in a second direction perpendicular to the first direction. The method may further include: catching, by the first and second tubing catchers, the tubing of the stethoscope; and holding, by the tubing holder of the stethoscope holder, the tubing of the stethoscope.

### DRAWINGS

FIG. 1 is a perspective view of a stethoscope holder according to some embodiments.

FIG. 2 is a front view of a stethoscope holder according to some embodiments.

FIG. 3A is a top view of a stethoscope holder according to some embodiments.

FIG. 3B is a bottom view of a stethoscope holder according to some embodiments.

FIG. 4A is a rear view of a stethoscope holder according to some embodiments.

FIG. 4B is a rear view of a stethoscope holder according to some embodiments when a clip is rotated.

FIG. 5 is a perspective view showing a use of a stethoscope holder according to some embodiments.

FIG. 6 is a flowchart showing use of a stethoscope holder according to some embodiments.

FIGS. 7A-7D and 8 are front views of a stethoscope holder according to some embodiments.

FIG. 9 is a flowchart showing use of a stethoscope holder according to other embodiments.

FIG. 10 is a perspective view of a stethoscope holder according to other embodiments.

FIG. 11 is an exploded view of a stethoscope holder according to other embodiments.

FIG. 12 is a rear view of a stethoscope holder according to other embodiments.

### EMBODIMENTS

The description of illustrative embodiments according to principles of the present disclosure is intended to be read in connection with the accompanying drawings, which are to be considered part of the entire written description. In the description of embodiments of the disclosure disclosed herein, any reference to direction or orientation is merely intended for convenience of description and is not intended in any way to limit the scope of the present disclosure. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the disclosure are illustrated by reference to the exemplified embodiments. Accordingly, the disclosure expressly should not be limited to such exemplary embodiments illustrating some possible non-limiting combination of features that may exist alone or in other combinations of features; the scope of the disclosure being defined by the claims appended hereto.

This disclosure describes the best mode or modes of practicing the disclosure as presently contemplated. This description is not intended to be understood in a limiting sense, but provides an example of the disclosure presented solely for illustrative purposes by reference to the accompanying drawings to advise one of ordinary skill in the art of the advantages and construction of the disclosure. In the various views of the drawings, like reference characters designate like or similar parts.

It is important to note that the embodiments disclosed are only examples of the many advantageous uses of the innovative teachings herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality.

Some embodiments of the present disclosure can provide a stethoscope holder. In one example, such a stethoscope holder can reduce or eliminate the need of a stethoscope being worn around the user's neck. As an example, the stethoscope holder can be worn on the user's waist or attached to a belt, with a clip of the stethoscope holder. Thus, some embodiments of the stethoscope holder of the present disclosure may reduce or avoid neck pain that may be caused due to wearing of a stethoscope around the user's neck.

In addition, in some embodiments, the stethoscope holder of the present disclosure may include a tubing holder and first and second tubing catchers. The tubing holder may hold a tubing of a stethoscope and the first and second tubing catchers may catch the tubing of the stethoscope. Therefore, the stethoscope holder of the present disclosure may more stably keep a stethoscope in place.

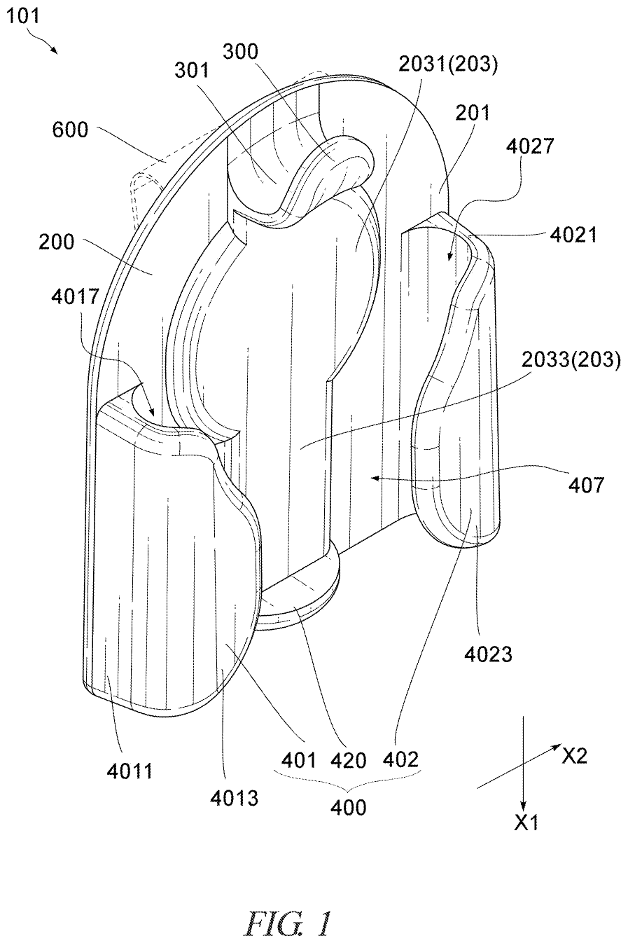

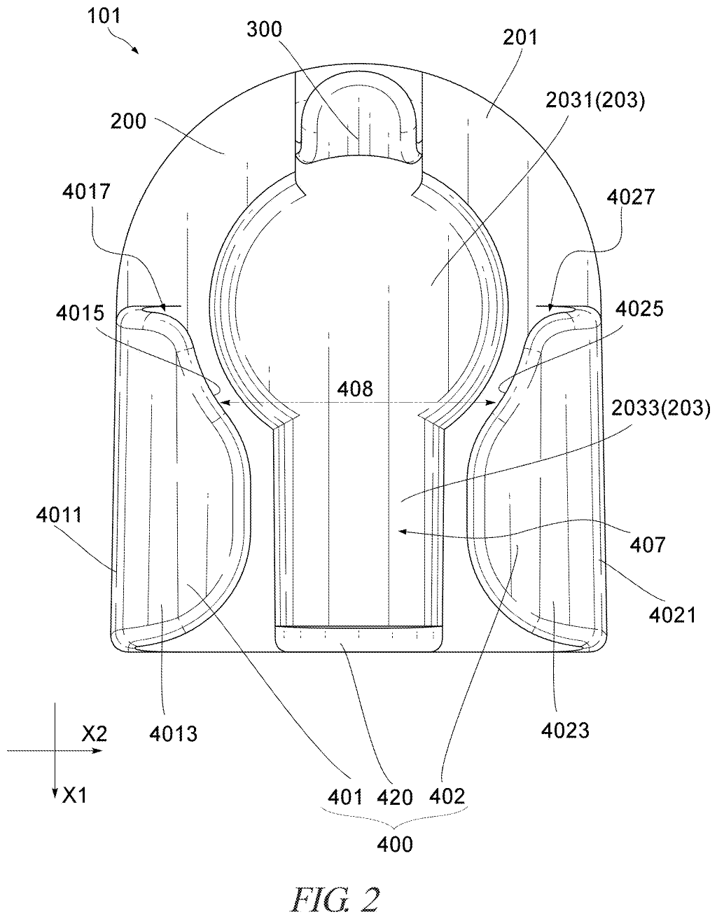

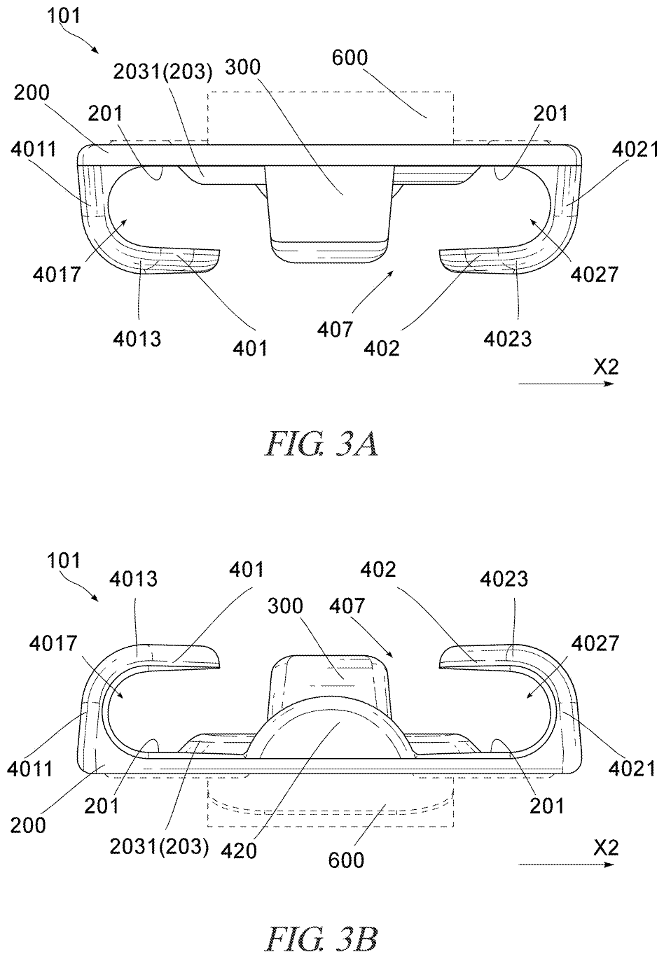

FIG. 1 is a perspective view of a stethoscope holder according to some embodiments. FIG. 2 is a front view of a stethoscope holder according to some embodiments. FIG. 3A is a top view of a stethoscope holder according to some embodiments. FIG. 3B is a bottom view of a stethoscope holder according to some embodiments.

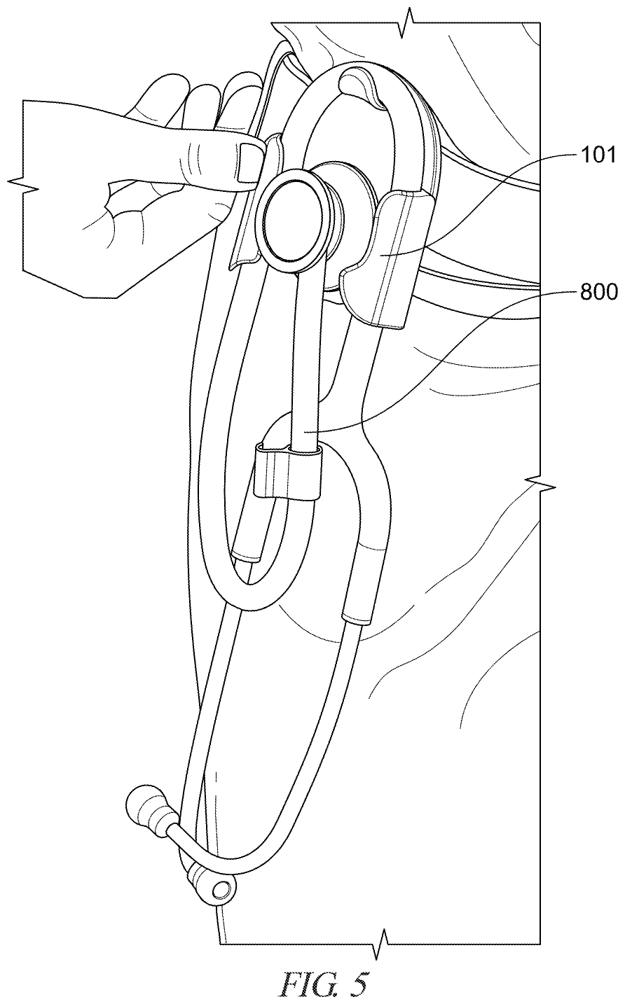

A stethoscope holder 101 in the present disclosure may be configured hold a stethoscope 800 (See FIG. 5). With reference to FIGS. 1-3B, the stethoscope holder 101 may include a base 200, a tubing holder 300, and a chestpiece holder 400. The base 200, the tubing holder 300, and the chestpiece holder 400 may be made of resin, rubber, metal, polymer, glass, ceramic, combination thereof, or other type of material. In some embodiments, the base 200, the tubing holder 300, and the chestpiece holder 400 may be formed integrally. In other embodiments, the base 200, the tubing holder 300, and the chestpiece holder 400 may be formed by a plurality of separate components.

With reference to FIGS. 1-3B, the tubing holder 300 may be formed on the base 200. In the illustrated example, the tubing holder 300 may be formed at the top of the base 200, but the location of the tubing holder 300 is not limited to this example, and may be any other location. In some embodiments, the tubing holder 300 may extend from the base 200. The tubing holder 300 may be configured to hold a tubing of the stethoscope 800. In the illustrated example, the tubing holder 300 may be a hook that has a curved surface 301. The curved surface 301 may be configured to hold the tubing of the stethoscope 800.

With reference to FIGS. 1-3B, the chestpiece holder 400 may be formed on the base 200. The chestpiece holder 400 may be located in a first direction X1 side of the tubing holder 300. The chestpiece holder 400 may be configured to hold a chestpiece of the stethoscope. In some embodiments, the chestpiece holder 400 may be configured to hold various types of chestpieces, and, in one example, the chestpiece holder 400 may be configured to hold chestpieces of adult and pediatric stethoscopes. In some embodiments, the chestpiece holder 400 may include first and second tubing catchers 401 and 402 and a chestpiece support 420. In one embodiment, the chestpiece support 420 may be provided with an upstanding lip or edge (not shown) to more securely retain a chestpiece that is seated on the support 420 against the base 200.

The chestpiece holder 400 may be provided with a gap 407. The gap 407 may be located between the first and second tubing catchers 401 and 402 in a second direction X2 perpendicular to the first direction X1. The chestpiece holder 400 may include various configurations, and some examples of the chestpiece holder 400 are described below. However, the configuration of the chestpiece holder 400 is not limited to these examples.

Each of the first and second tubing catchers 401 and 402 may be configured to catch the tubing of the stethoscope 800. Optionally, the first and second tubing catchers 401 and 402 may be configured to guide or hold a chestpiece of the stethoscope. The first tubing catcher 401 may include a first side wall 4011 and a first front wall 4013, and the second tubing catcher 402 may include a second side wall 4021 and a second front wall 4023.

In the illustrated example shown in FIGS. 1-3B, the first side wall 4011 may extend from the base 200 and may extend in the first direction X1. The first front wall 4013 may extend from the first side wall 4011 toward the second front wall 4023. The first front wall 4013 and the first side wall 4011 may form a first tubing path 4017. The first tubing path 4017 may be opened in the first direction X1 (e.g., the first tubing path 4017 may be opened downward in the illustrated example in FIG. 2). In the illustrated example shown in FIGS. 1-3B, the second side wall 4021 may extend from the base 200 and may extend in the first direction X1. The second side wall 4021 and the first side wall 4011 may be spaced apart from each other in the second direction X2. The second front wall 4023 may extend from the second side wall 4021 toward the first front wall 4013. The second front wall 4023 and the second side wall 4021 may form a second tubing path 4027. The second tubing path 4027 may be opened in the first direction X1 (e.g., the second tubing path 4027 may be opened downward in the illustrated example in FIG. 2). The first and second front walls 4013 and 4023 form the gap 407 between the first and second front walls 4013 and 4023.

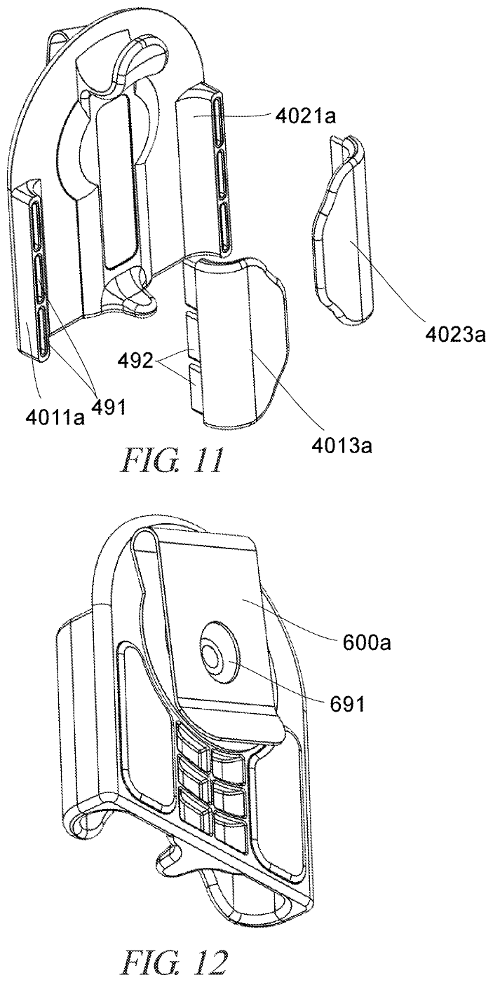

In some embodiments, the first front wall 4013 and the first side wall 4011 may be formed integrally, and the second front wall 4023 and the second side wall 4021 may be formed integrally. In other embodiments, as shown in FIG. 11, the first front wall 4013a and the first side wall 4011a may be formed from separate components, and the second front wall 4023a and the second side wall 4021a may be formed from separate components. In some examples as shown in FIG. 11, the first front wall 4013a may be interchangeable. In the illustrated example, the first front wall 4013a may be attached to the first side wall 4011a. In some embodiments, the first front wall 4013a may be interchangeable, but be permanently attached to the first side wall 4011a, for example, by ultrasonic welding during an assembly process of the stethoscope holder. In some embodiments, the first front wall 4013a may be removably attached to the first side wall 4011a, and be interchangeable with another front wall, such that, after purchase, tubing catchers can be easily changed to fit different stethoscope heads. The first front wall 4013a may be attached to the first side wall 4011a, for example, by using at least one screw coming in from the backside of the stethoscope holder. In some examples, one of the first front wall 4013a and the first side wall 4011a may be fitted into the other one of the first front wall 4013a and the first side wall 4011a. In some embodiments, one of the first front wall 4013a and the first side wall 4011a may include at least one protrusion 492, and the other one of the first front wall 4013a and the first side wall 4011a may include at least one recess 491 into which the protrusion 492 is fitted. However, the first front wall 4013a may be attached to the first side wall 4011a in a way different from the example shown in FIG. 11. In some examples as shown in FIG. 11, the second front wall 4023a may be interchangeable. In the illustrated example, the second front wall 4023a may be attached to the second side wall 4021a in the same manner as the first front wall 4013a is attached to the first side wall 4011a. However, the second front wall 4023a may be attached to the second side wall 4021a in a way different from the example shown in FIG. 11. By using the interchangeable first front wall 4013a and second front wall 4023a, the height or design of tubing catchers can be easily changed to fit different stethoscope heads.

With reference to FIG. 2, the first front wall 4013 may include a first edge 4015, and the second front wall 4023 may include a second edge 4025. In some embodiments, a distance 408 in the second direction X2 between the first and second edges 4015 and 4025 may be decreased as the first and second edges 4015 and 4025 are distant from the tubing holder 300 in the first direction X1. Thus, the first and second edges 4015 and 4025 may more securely retain the chestpiece of the stethoscope. However, the relationship between the first and second edges 4015 and 4025 is not limited to this example.

With reference to FIGS. 1 and 2, the chestpiece support 420 may extend from the base 200. The chestpiece support 420 may be configured to support a chestpiece of the stethoscope 800. In one example, chestpiece support 420 may include a top surface that is configured to support the chestpiece. In addition, in some embodiments, the chestpiece support 420 may include an edge that is configured to guide a part (e.g., which is close to the chestpiece) of the tubing of the stethoscope. In one embodiment, the chestpiece support 420 may be provided with an upstanding lip or edge to more securely retain a chestpiece that is seated on the support 420. The gap 407 may be located between the tubing holder 300 and the chestpiece support 420 in a front view. In some embodiments, the chestpiece guide 400 does not include the chestpiece support 420, and, for example, the first tubing catcher 401 and the second tubing catcher 402 may be configured to support a chestpiece of a stethoscope.

With reference to FIGS. 1-3B, the base 200 may include a tubing support surface 201, and a raised portion 203 raised from the tubing support surface 201. The tubing support surface 201 may include a part that overlaps with the chestpiece holder 400 in the front view. The raised portion 203 may include a part located differently from the chestpiece holder 400 in the front view. In some embodiments, the raised portion 203 may include a circular portion 2031 and an extension 2033. The extension 2033 may extend from the circular portion 2031 in the first direction X1. The base 200 is not limited to this example, and may include any other configuration. For example, the whole front surface of the base 200 may be flat.

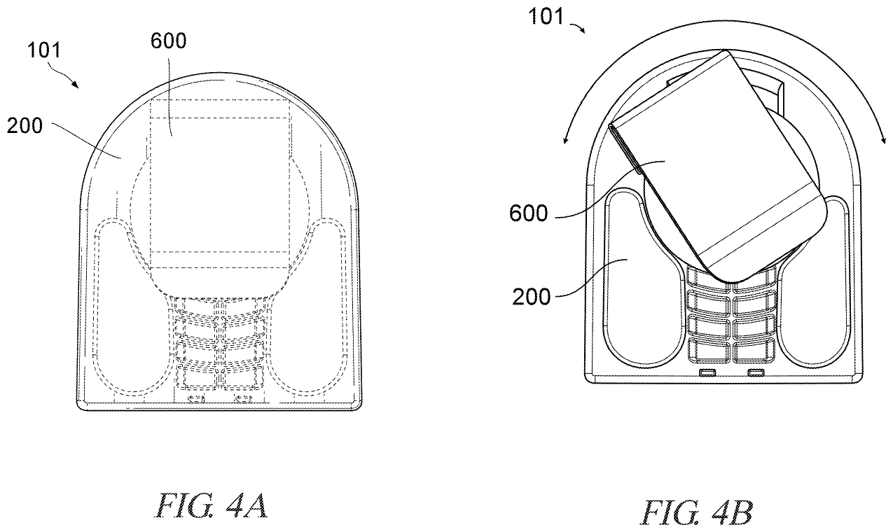

FIG. 4A is a rear view of a stethoscope holder according to some embodiments.

As shown in FIG. 4A, the stethoscope holder 101 may further include a clip 600. The clip 600 may be formed on a back side of the base 200. Optionally, as shown in FIG. 4B, the clip 600 may be rotatable relative to the base 200. The clip 600 allows the stethoscope holder 101 to be worn on a user's waist or attached to a belt. See FIG. 5. Examples of the clip 600 include a metal back clip such that the clip 600 can easily slide on a user's waist or a belt. As shown in FIG. 12, a stethoscope holder may include a clip 600a that has a different configuration than the clip of FIG. 4A. As shown in FIG. 12, the clip 600a may have an opening 691. Configurations of a clip are not limited to these examples of FIG. 4A and 12, and a clip of a stethoscope holder may have various configurations.

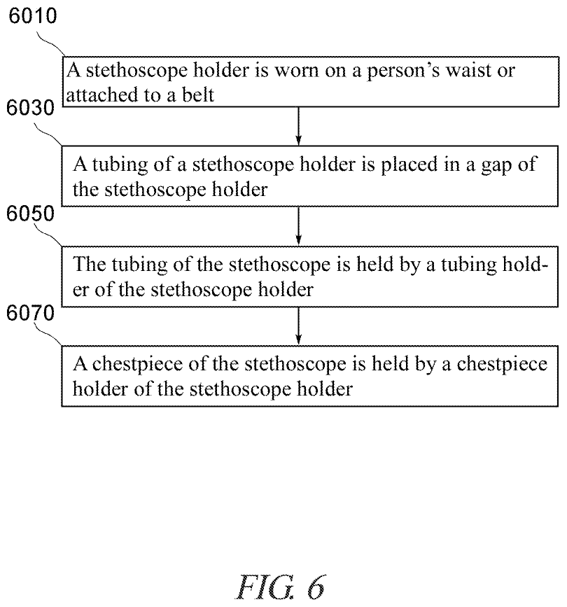

FIG. 6 is a flowchart showing use of the stethoscope holder 101 according to some embodiments.

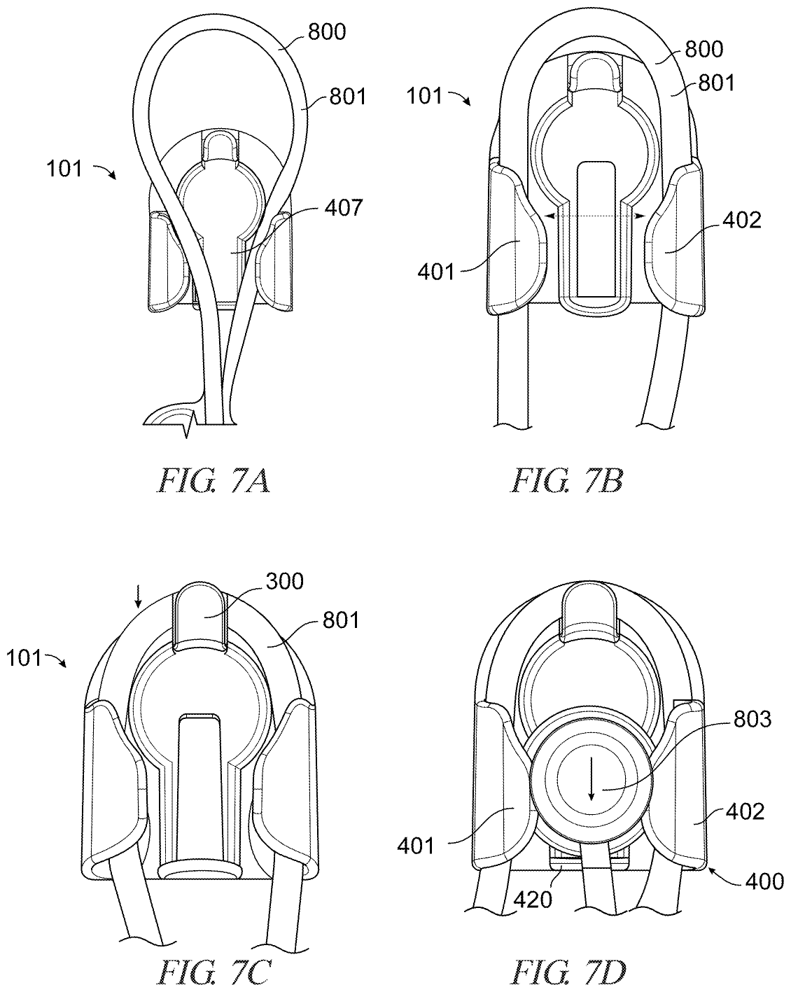

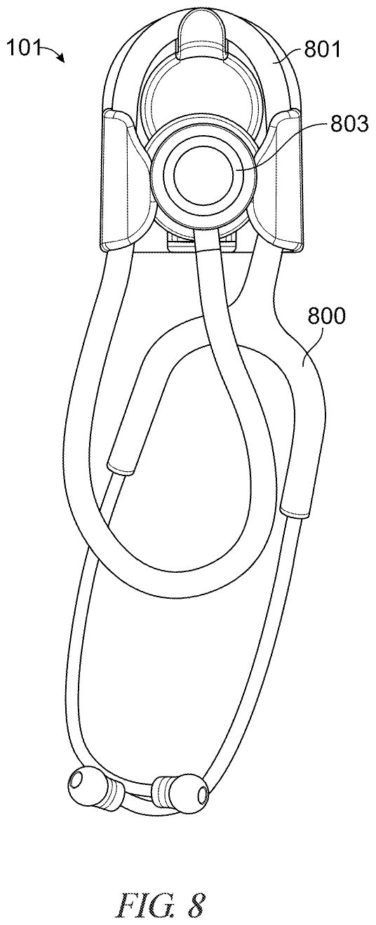

With reference to FIG. 6, in 6010, the stethoscope holder 101 may be worn on a user's waist or attached to a belt with the clip 600. Then, as shown in FIGS. 6 and 7A, in 6030, a tubing 801 of a stethoscope 800 may be placed in the gap 407 of the stethoscope holder 101. Then, as shown in FIGS. 6 and 7B, in 6050, the tubing 801 of the stethoscope 800 may be caught by the first and second tubing catchers 401 and 402. Then, as shown in FIGS. 6 and 7C, in 6050, the tubing 801 of the stethoscope 800 may be held by the tubing holder 300 of the stethoscope holder 101. Then, as shown in FIGS. 6, 7D, and 8, in 6070, a chestpiece 803 of the stethoscope 800 may be held by the chestpiece holder 400 of the stethoscope holder 101.

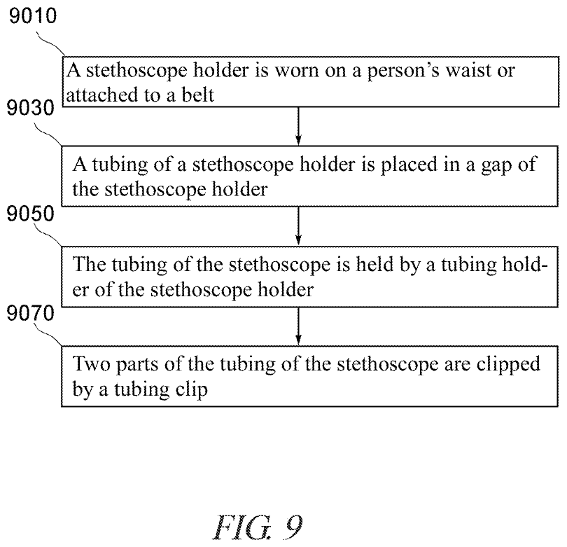

FIG. 9 is a flowchart showing use of the stethoscope holder 101 according to other embodiments.

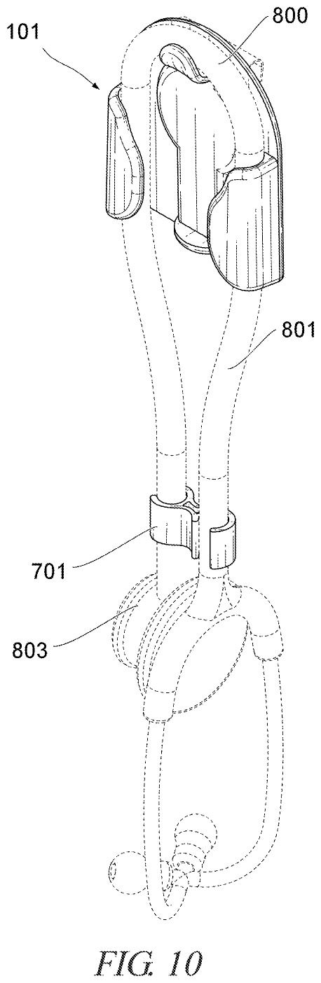

Processes 9010, 9030, and 9050 in FIG. 9 may be conducted in the same manner as the processes 6010, 6030, and 6050 in FIG. 6, which are described above. After 9050, as shown in FIGS. 9 and 10, in 9070, two parts of the tubing 801 of the stethoscope 800 may be clipped by a tubing clip 701.

According to the present disclosure, the stethoscope holder 101 can reduce or eliminate the need of a stethoscope being worn around a user's neck. As an example, the stethoscope holder 101 can be worn on the user's waist or attached to a belt, with the clip 600 of the stethoscope holder 101. Thus, some embodiments of the stethoscope holder 101 may reduce or avoid neck pain due to wearing of a stethoscope around the user's neck.

According to the present disclosure, the tubing holder 300 may hold the tubing 801 of the stethoscope 800 and the first and second tubing catchers 401 may catch the tubing 801 of the stethoscope 800. Therefore, the stethoscope holder 101 may more stably keep the stethoscope 800 in place. In some embodiments, for example as shown in FIG. 8, the chestpiece holder 400 may hold the chestpiece 803 of the stethoscope 800, such that the stethoscope holder 101 may more stably hold the stethoscope 800. In other embodiments, for example as shown in FIG. 10, the two parts of the tubing 801 of the stethoscope 800 may be clipped together by the tubing clip 701, such that the stethoscope holder 101 and the tubing clip 701 may provide stabler holding of the stethoscope 800.

Further, in some embodiments, the clip 600 may be rotatable relative to the base 200. The rotation of the clip 600 can prevent the stethoscope holder 101 from getting out of the way, for example, when the user sits on a chair.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All examples and conditional language recited herein are intended for pedagogical purposes to aid the reader in understanding the principles of the disclosure and the concepts contributed by the inventor to furthering the art, and are to be construed as being without limitation to such specifically recited examples and conditions. Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A stethoscope holder, comprising:
a tubing holder; and
a chestpiece holder located in a first direction side of the tubing holder, the chestpiece holder including first and second tubing catchers, the chestpiece holder being provided with a gap between the first and second tubing catchers in a second direction perpendicular to the first direction.

2. The stethoscope holder of claim 1, wherein the first tubing catcher includes a first side wall, and
the second tubing catcher includes a second side wall, the first and second side walls spaced apart from each other in the second direction.

3. The stethoscope holder of claim 2, wherein each of the first and second side walls extends in the first direction.

4. The stethoscope holder of claim 2, wherein the first tubing catcher includes a first front wall, and the second tubing catcher includes a second front wall,
the first front wall extends from the first side wall toward the second front wall, and the second front wall extends from the second side wall toward the first front wall, and
the first and second front walls form the gap between the first and the second front walls.

5. The stethoscope holder of claim 4, wherein the first front wall includes a first edge, and the second front wall includes a second edge, and
a distance in the second direction between the first and second edges is decreased as the first and second edges are distant from the tubing holder in the first direction.

6. The stethoscope holder of claim 4, wherein the first side wall and the first front wall form a first tubing path that is opened in the first direction, and
the second side wall and the second front wall form a second tubing path that is opened in the first direction.

7. The stethoscope holder of claim 4, wherein the first front wall is removably attached to the first side wall, and the second front wall is removably attached to the second side wall.

8. The stethoscope holder of claim 1, further comprising a base,
wherein the tubing holder and the chestpiece holder are formed on the base.

9. The stethoscope holder of claim 8, wherein the base includes a tubing support surface, and a raised portion raised from the tubing support surface, and
the tubing support surface includes a part that overlaps with the chestpiece holder in a front view, and the raised portion includes a part located differently from the chestpiece holder in the front view.

10. The stethoscope holder of claim 9, wherein the raised portion includes:
a circular portion; and
an extension extending from the circular portion in the first direction.

11. The stethoscope holder of claim 8, wherein the chestpiece holder comprises a chestpiece support extending from the base, and
the gap is located between the tubing holder and the chestpiece support in a front view.

12. The stethoscope holder of claim 8, further comprising a clip formed on a back side of the base.

13. The stethoscope holder of claim 12, wherein the clip is rotatable relative to the base.

14. The stethoscope holder of claim 12, wherein the clip allows the stethoscope holder to be worn on a user's waist or attached to a belt.

15. A method of using a stethoscope holder, comprising:
placing, in a gap of the stethoscope holder, a tubing of the stethoscope holder,
the stethoscope holder including: a tubing holder; and a chestpiece holder located in a first direction side of the tubing holder,
the chestpiece holder including first and second tubing catchers, and
the gap being located between the first and second tubing catchers in a second direction perpendicular to the first direction;
catching, by the first and second tubing catchers, the tubing of the stethoscope; and
holding, by the tubing holder of the stethoscope holder, the tubing of the stethoscope.

16. The method of claim 15, further comprising holding, by the chestpiece holder of the stethoscope holder, a chestpiece of the stethoscope.

17. The method of claim 15, further comprising clipping, by a tubing clip, two parts of the tubing of the stethoscope.
